(19) United States
(12) Patent Application Publication
(10) Pub. No.: US 2026/0334476 A1
(43) Pub. Date: Oct. 29, 2026

(54) MICROCAPSULE COMPRISING A BACTERIOPHAGE

(71) Applicants: LESAFFRE ET COMPAGNIE, PARIS (FR); INSTITUT NATIONAL DE RECHERCHE POUR L'AGRICULTURE, L'ALIMENTATION ET L'ENVIRONNEMENT, PARIS (FR)

(72) Inventors: Mickaël BOYER, MARCQ EN BAROEUL (FR); Antoine DREVELLE, BORDEAUX (FR); Juan Sebastian LOPEZ FERNANDEZ, PARIS (FR); Camille PROFIZI, NANTES (FR); Clara TORRES BARCELÓ, MONTPELLIER (FR)

(21) Appl. No.: 18/879,655

(22) PCT Filed: Jun. 29, 2023
(86) PCT No.: PCT/EP2023/067856

§ 371 Date: Dec. 27, 2024

(30) Foreign Application Priority Data
Jun. 30, 2022 (EP) 22305955.1

(51) Int. Cl. A01N 63/40; A01N 25/04; A01P 1/00; A23B 2/783; A61K 9/50; A61K 35/76; A61P 31/04

(52) U.S. Cl. CPC A01N 63/40; A01N 25/04; A01P 1/00; A23B 2/783; A61K 9/501; A61K 35/76; A61P 31/04

(57) ABSTRACT

The present invention relates to the field of protection against bacterial infectious diseases by means of compositions comprising bacteriophages. It concerns in particular an antibacterial composition comprising: at least one bacteriophage chosen from the Podoviridae, Myoviridae and Siphoviridae, as an active agent in a liquid core, a gelled shell encapsulating the liquid core comprising said bacteriophage and comprising dispersed colloidal particles of UV screening agent, in which the UV screening agent is not a soluble antioxidant compound.

The present invention relates to the field of protection against bacterial infectious diseases by means of compositions comprising bacteriophages.

Pathogenic bacteria are able to infect various hosts: human, animal, plant. Antibacterials can treat bacterial infections either by eliminating bacteria (bactericidal compounds) or by blocking the multiplication thereof (bacteriostatic compounds). Schematically, antibacterials can be antibiotics of plant or fungal origin, of bacterial origin or of fossil origin (via extraction or chemical synthesis). Antibacterials can also be of animal origin or even of viral origin. In this latter case, the viruses are phages or bacteriophages which infect bacteria.

Bacteria are capable of mutating and thereby of adapting to their environment and to antibacterials. It is for this reason that resistance to antibiotics has become a major issue, and the recognition of this resistance has led to considering alternative antibacterial treatments such as bacteriophages. Also, bacteriophages rapidly adapt to bacterial mutations without however having a negative effect on commensal flora. Finally, bacteriophages do not infect the eukaryote cells and are therefore harmless for use in humans, animals, fish, or plants.

In the sphere of human health, the administering of bacteriophages as early as the 1960s (e.g. the phage [symbol] X174 belonging to the Micovirida and infecting Escherichia coli) showed that they are safe to use, and have particular application in the event of antibiotic resistance or to complement an antibiotherapy. Without being limited thereto, mention can be made of ENT-related infections with Pseudomonas aeruginosa such as otitis, and infections of the intestinal system with Escherichia coli.

In the sphere of animal health and nutrition where the trend is towards reducing the use of antibiotics, there is a true advantage in the use of bacteriophages. In pisciculture for example, for the management of rainbow trout or salmon pathogens such as Flavobacterium psychrophilum, or the Yersinia ruckeri pathogen which infects salmon. In poultry breeding, phage cocktails are used against Salmonella or Clostridium perfringens, such as those developed by Intralytix Inc.

In the sphere of plant health where bacterial infections are responsible for major economic losses, most existing biocontrol methods are based on the use of copper, but the toxicity and potential side effects thereof have led to the search for other biocontrol solutions including the use of bacteriophages.

Finally, in the agri-food sector, bacteriophages can be used to ensure food safety through the ability thereof to limit the development of pathogens at food processing steps or on surfaces in contact with foods. Not limited thereto, Listeria, Shigella, Salmonella, Escherichia coli or Campylobacter jejuni are priority targets in the agri-food industry.

One of the difficulties when using bacteriophages is that they are fragile and sensitive to the environment. They have walls that are solely composed of proteins, these walls being thinner and more fragile than those of bacteria. More particularly, the extreme pH of some soils or even UV radiation. for example the sun rays, are factors to be taken into account.

Improving the stability and maintaining the viability of phages over time is a major challenge for the envisaging of large-scale use thereof. Spray-drying or freeze-drying technologies exist (Matinkhoo et al., 2011, J Pharm Sci; 100(12):5197-205; Jones et al., 2012, Bacteriophage 2: 208-214; Vandenheuvel et al, 2013, Eur J Pharm Biopharm; 84(3):578-82; Leung et al., 2016, Pharmaceutical research, 33(6), 1486-1496; Leung et al., 2017, International journal of Pharmaceutics, 521(1-2), 141-149). Encapsulating technologies intended to improve stability at the time of storage, transport and application have been tested.

When searching for solutions intended to protect bacteriophages against external stresses of pH or UV radiation type during storage or application, the Applicant experimented the co-drying of bacteriophages and yeasts as explained in application FR2110984.

Surprisingly, the encapsulation of bacteriophages used as active agent within a shell comprising dispersed colloidal particles of UV screening type, allows the maintaining of phage viability and antibacterial action. Phages thus encapsulated provide efficient control over bacterial pathogens, advantageously bacterial pathogens directed against humans, animals, or plants.

The invention therefore relates to an antibacterial composition comprising at least one bacteriophage chosen from the Podoviridae, Myoviridae and Siphoviridae, as active agent in a liquid core, said liquid core comprising the bacteriophage encapsulated in a gelled shell comprising dispersed colloidal particles of UV screening agent, in which the UV screening agent is not a soluble antioxidant compound.

### DETAILED DESCRIPTION

The terms phages or bacteriophages are used indifferently. Phages or bacteriophages are viruses which infect bacteria in a manner that is highly specific and without any significant side effect in the human, animal, or plant host. The antibacterial composition subject of the present invention is therefore directed against any potentially pathogenic bacterium, whether the host is a human, animal, or plant. By antibacterial action, it is understood lysis of the bacterium after it has been infected by the bacteriophage.

In one embodiment, the targeted pathogens are bacteria likely to infect plants and/or harvest products. Bacterial diseases of plants cause great losses in agricultural crops and vegetable crops. Few treatments are available and many are copper-based. Iriarte et al. (Bacteriophage, 2012; 2 (4): 215-224) showed that a phage is able to penetrate inside the plant moving from the roots to the leaves and persisting within the plant for up to 7 days.

With respect to the above-ground parts of plants, the targeted pathogens are chosen from among: Erwinia spp. or more specifically Erwinia amylovora, Xanthomonas spp. or more specifically Xanthomonas axonopodis var. citri the host being the citrus in which this pathogen is responsible for the disease commonly called Citrus greening, Xylella spp. or more specifically Xylella fastidiosa able to infect about 600 plant species belonging to more than 80 different botanical families: grapevine, citrus fruits, fruit trees, almond trees, olive trees, cherry trees, coffee plants, avocado trees, alfalfa, oleander, oak, maple etc. the disease affecting the grapevine is called Pierce's disease.

For soil diseases, for which application via a drip system is necessary or application to seeds, the targeted pathogens are chosen from among Pseudomonas spp. or more specifically Pseudomonas syringae pv. tomato which is responsible for the disease called bacterial speck in the host tomato plant, Ralstonia spp. or more specifically Ralstonia solanacearum for which the host is also the tomato plant and is responsible for the disease known as bacterial wilt.

Regarding the products of harvested plants for which application is directly onto the product, the pathogens that are particularly targeted are the species Pectobacterium and Dickeya likely to infect the potato plant.

Erwinia amylovora is a pathogenic plant bacterium responsible for fire blight which is a contagious disease affecting apple trees, pear trees and other plants in the Rosacae family (Momol M T; Aidwinckle H S, 2000. Genetic diversity and host range of Erwinia amylovora. In: Vanneste J L, ed. Fire blight the disease and its causative agent, Erwinia amylovora. Wallingford, Oxon UK: CABI Publishing, 55-72: Zwet T van der; Keil H L, 1979. Fire blight, a bacterial disease of Rosaceous plants. Agriculture Handbook, Science and Education Administration USDA. Beltsville, USA: USDA, No. 510:200 pp). Screening of a collection of 33 phages infecting the species Erwinia allowed the isolating for example of five phages in the Myoviridae family which are able to infect a broad range of bacterial strains of Erwinia amylovora. Said phages, respectively called BPH1, 31Dx, 26C, P2 and 2By, were used for the encapsulation tests of the antibacterial compositions of the invention.

In one embodiment, the targeted pathogens are bacteria likely to infect humans. Among these, but not limited thereto, are the bacterial strains Escherichia coli, Listeria monocytogenes, Campylobacter jejuni, Staphylococcus aureus, Clostridium perfringens or strains of the genus Salmonella.

In one embodiment, the targeted pathogens are bacteria likely to infect animals such as Escherichia coli, Salmonella, Campylobacter, Staphylococcus, Flavobacterium psychrophilum, Yersinia ruckeri, Clostridium perfringens. The animals to be treated are not only bovines but also sheep, swine, fowl, fish, crustaceans etc.

In one embodiment, the targeted pathogens are pathogens against which the agri-food industry seeks protection for dairy products, meat or fish: Listeria, Salmonella, Shigella, Escherichia coli, Campylobacter jejuni etc.

Advantageously, encapsulation allows the antibacterial composition of the invention to maintain its antibacterial properties such as demonstrated by the experimental results given in the examples below. To demonstrate the viability of a phage and for phage enumeration, it is essential that said phage should remain effective against the pathogen against which it is directed.

The capsules used in the antibacterial composition of the invention have a shell prepared from a liquid solution comprising a biopolymer having gelling properties and comprising dispersed colloidal particles of UV screening agent, wherein the UV screening agent is not a soluble antioxidant compound.

The microcapsules used in the antibacterial composition of the invention allow confining of the bacteriophages but not the immobilisation thereof.

Microcapsules are well-known to persons skilled in the art. They can be formed by different techniques and can have different shell compositions. Typically, the microcapsules used in the invention are produced following the fabrication method described in French patent FR 2939012 and international application t WO2010063937 A1.

They are formed of a liquid core comprising at least one phage forming the active agent of the antibacterial composition of the invention. This liquid core may comprise a mixture of different phages.

The liquid core is encapsulated by a gelled, substantially solid casing called the shell. In particular, said shell is solid. Preferably, the shell of the microcapsules is chiefly composed of a biopolymer having gelling properties. In particular, said shell is composed of said biopolymer having gelling properties. This biopolymer in majority proportion in the shell is the main biopolymer. Said biopolymers having gelling properties are for example alginate, gellan gum, xanthan gum, pectin, chitosan, agar or carragheenin. Preferably, the shell of the microcapsules forming the antibacterial composition of the invention is chiefly composed of alginate, in particular said shell is composed of alginate.

When in hydrated form, for example in suspension in an aqueous solution, the microcapsules of the antibacterial composition of the invention have a mean diameter of between 50 and 4000 μm, preferably between 100 and 2000 μm, more particularly between 200 and 1000 μm, advantageously between 200 and 600 μm. This mean diameter can be measured with various techniques known to skilled persons such as particle size analysis based on laser light diffraction, fractionation by screening or optical microscopy imaging. They can also be in dehydrated form. Preferably dehydration is partial and the hydrated microcapsules preferably have a moisture level of less than 10%, measured by a moisture analyzer after dehydration.

Preferably, the microcapsules of the invention are stand-alone i.e. they are not included in another structure such as a film, bead, gel or encapsulated a second time, but they are in direct contact with their surrounding medium, typically a liquid (if in suspension for example) or a gas.

By colloidal particles of UV screening agent, it is meant particles of mean size between 10 nm and 10 μm, more preferably between 100 nm and 5 μm. In general, the smaller the particles the more efficient they are against UV rays. These screening agents can also opacify the microcapsules.

In the context of the present invention, the UV screening agent is not a soluble antioxidant compound. Such compounds tend to degrade more rapidly when exposed to UV rays. The UV screening agent used in the microcapsules forming the antibacterial composition of the invention can be organic or inorganic, or a mixture of organic and inorganic screening agent. Said UV screening agent is preferably chosen from among titanium oxide, carbon black, biochar, charcoal, latex, silica, clays, and mixtures thereof. Biochar is the product of biomass pyrolysis, used as soil amendment in agriculture. The clays used as UV screening agents are for example talc, or kaolin, and mixtures thereof. Kaolin is a type of clay also used to protect some crops against insect pests in agriculture.

Preferably, the UV screening agent used in the microcapsules forming the antibacterial composition of the invention is biodegradable and/or comestible and/or pharmaceutically acceptable.

By «dispersed particles of UV screening agent», it is meant particles that are dispersed preferably in the shell of the microcapsule, to provide a homogeneous anti-UV effect on the surface of the microcapsule. Preferably, the core of the capsule of the invention does not comprise particles of UV screening agent. In particular, the concentration of UV screening agent is between 0.05 and 30% v/v, more particularly between 0.1 and 20% v/v, relative to the total volume of the shell.

Preferably, in the methods and uses of the invention, the microcapsules are used as such i.e. they are not included in another structure (such as a film, a bead, gel or encapsulated a second time), but they are used directly optionally in suspension in a liquid.

The invention also relates to a method for preventing and/or treating plants against at least one pathogenic bacterium, comprising the steps of:
providing an antibacterial composition comprising at least one bacteriophage as active agent against said at least one pathogenic bacterium, said bacteriophage being protected against UV rays in a microcapsule comprising colloidal particles of UV screening agent dispersed in a shell, wherein said UV screening agent is not a soluble antioxidant agent
applying said composition to the above-ground parts of the plant.

In particular, said at least one bacteriophage is chosen from among Podoviridae, Myoviridae and Siphoviridae.

In one embodiment, the harvested products of said plants are subjected to the antibacterial prevention and/or treatment.

In one embodiment, the antibacterial composition can also be applied by ground-spreading. In another embodiment, the antibacterial composition can be applied around seeds, in particular but not only by coating or filming. In another embodiment, application can be performed on the products after harvesting.

In another embodiment, the invention relates to the preferably non-therapeutic use of an antibacterial composition of the invention for animal and/or human nutrition and/or nourishment. In this embodiment, the phages used are preferably not therapeutic phages. In other words, said phages are of interest from a nutritional or nourishment viewpoint but do not allow the preventing or treating of the diseases of the animal or individual consuming these phages.

By «nourishment», it is meant usual or frequent food intake. The antibacterial composition may contain varied phages allowing the properties of a food product to be improved, which can include bioavailability or storage time.

By «nutrition», it is meant the intake of a food supplement, that is most often occasional or for a cure period, typically to prevent or compensate for a deficiency. The antibacterial composition of the invention can contain varied phages which can improve nutrient intake by improving the bioavailability thereof, or can modify intestinal flora.

The invention also relates to a pharmaceutical composition comprising an antibacterial composition of the invention.

The present invention also relates to an antibacterial composition or pharmaceutical composition of the invention for use thereof as antibacterial agent in a subject in need thereof, in particular as medicinal product, and more particularly for the treatment of a disease caused by a bacterium.

In this embodiment, the antibacterial composition of the invention advantageously comprises a phage chosen from among bacteriophages targeting pathogenic bacteria in Man or animal, and more particularly a bacteriophage targeting bacteria chosen from among Escherichia coli, Listeria monocytogenes, Campylobacter jejuni, Staphylococcus aureus, Clostridium perfringens or strains of the genus Salmonella.

The term “pharmaceutical composition” such as defined herein designates a mixture or solution comprising at least one therapeutic agent to be administered to a subject, for the prevention or treatment of a particular disease affecting the subject.

The pharmaceutical compositions such as defined herein therefore additionally and preferably comprise pharmaceutically acceptable excipients.

By “pharmaceutically acceptable”, it is meant herein compositions and molecular entities which do not produce secondary, allergic, or other adverse reactions when administered to a subject.

By “subject”, it is meant herein a living being, preferably a mammal and more particularly a human being.

By “therapeutically effective amount”, it is meant herein at the necessary doses and time periods to obtain the desired therapeutic result. This amount can vary as a function of factors such as the disease, extent of the disease, age, gender and weight of the subject, and the capability of the microcapsules to induce the desired therapeutic result. A therapeutically effective amount encompasses an amount for which any toxic or harmful effect is offset by the therapeutic beneficial effects. A therapeutically effective amount also encompasses an amount sufficient to impart a benefit, for example a clinical benefit.

Said pharmaceutical compositions are preferably adapted to the route of administration.

In one particular embodiment, the pharmaceutical compositions are adapted for oral, sublingual, buccal, intranasal, or topical administration.

As aforementioned, the invention also concerns an antibacterial composition or a pharmaceutical composition of the invention for use as medicinal product. The invention also concerns an antibacterial composition of the invention for use thereof for the formulating of pharmaceutical compositions.

For these uses, the antibacterial composition of the invention advantageously comprises a phage chosen from among bacteriophages targeting pathogenic bacteria of Man or animal, and more particularly a bacteriophage targeting bacteria chosen from among Escherichia coli, Listeria monocytogenes, Campylobacter jejuni, Staphylococcus aureus, Clostridium perfringens or strains of the genus Salmonella.

The use of an antibacterial composition of the invention allows facilitated storage, packaging, or preparation of the pharmaceutical compositions while preserving the active phages.

By “treatment” or “treat”, it is meant herein the partial or substantial attaining of one or more of the following results: partial or full reducing of the extent of the disease, improvement in a clinical symptom or indicator associated with the disease, the retarding, inhibiting, or preventing of progress of the disease. In particular, in the present invention, the disease to be treated is a disease caused by a bacterium, in particular those mentioned above.

The examples below are given for illustration purposes and cannot be construed as limiting the scope of the invention.

### EXAMPLES

### Phage Enumeration Method

The methods for demonstrating bacteriophage viability and enumeration necessarily use the bacteria targeted by the tested bacteriophages. In general, the enumeration methods of bacteriophages of unknown concentration are semi-solid agar overlay methods known to those skilled in the art.

A preculture of target bacteria was obtained in liquid LB medium (Luria Bertani broth). In some cases, the preculture step in liquid medium can be doubled to reach sufficient volume and concentration, said concentration being evaluated by OD measurement (optical density) at 600 nm.
Serial dilution of the phages to be enumerated was conducted in liquid LB medium. Contacting of the serial dilutions of the phages with the bacterial cultures was performed on LBA medium (Luria Bertani Agar) previously auditioned with semi-solid Luria Bertani Agar medium at 0.75% agar.
Read-out and enumeration of the lysis zones were carried out after incubation for 16 to 24 hours at 30 °C.

A Salmonella Newport strain was used to enumerate anti-Salmonella phages supplied by Intralytix Inc (Salmofresh™).

A Listeria monocytogenes strain was used to enumerate the anti-Listeria phages supplied by Intralytix Inc.
An Erwinia amylovora Ea4 strain supplied by INRAe was used to enumerate anti-Erwinia phages (phages BPH1, 31Dx, 26C, P2 and 2By).

### Example 1: Phage Encapsulation Test and Evaluation of Stability after Encapsulation

The above-mentioned phage BPH1 was encapsulated in a capsule without UV screening agent. Drying tests were performed with three different drying substrates. Viability and stability over time as a function of temperature were evaluated.

Among glycerol, SM-sucrose and trehalose tested as drying excipients, SM-sucrose is the excipient which gave the most acceptable drying yield.

The stability of the encapsulated phage was tested for one month at +4 °C, +25 °C and +40 °C respectively. The titer of the encapsulated phage, in liquid form, decreases very little at +4 °C or +25 °C. The titer is reduced by about 2 log at +40 °C. In solid form i.e. after drying with SM-sucrose as excipient, the titer remains substantially the same irrespective of the storage temperature, with a decrease of less than 1 log at +40 °C.

### Example 2: Protection of Anti-Erwinia Phages in a Capsule Comprising a UV Screening Agent

The above-mentioned phages BPH1, 31Dx, 26C, P2 and 2By were encapsulated in a shell comprising biochar as anti-UV screening agent. Phages alone, capsules with or without biochar containing phages were exposed in liquid form to UV radiation.

The phages were deposited with the collection of microorganism cultures: Collection Nationale de Cultures de Microorganismes, housed at Institut Pasteur, 25 rue du docteur Roux, 75015 Paris, France, in accordance with the Budapest treaty:
BPH1 deposited on 17 Dec. 2021 under number 1-5803,
31Dx deposited on 6 May 2022 under number 1-5845,
26C deposited on 6 May 2022 under number 1-5846,
P2 deposited on 6 May 2022 under number 1-5847, and
2By deposited on 6 May 2022 under number 1-5848.

The protocol for exposure to UV radiation was the following:
recovery of a homogeneous sample of capsules of size 100 μm using sterile cell strainers;
sampling 3 g of wet capsules or else 1 g of dry capsules respectively, sieved into a sterile Petri dish;
addition of 0.1M NaCl buffer in 1:10 dilution with sterile physiological saline;
removal of control before exposure;
exposure to UV rays with a lamp previously preheated as is required, at an intensity of 105 mW/cm².
   The Petri dishes are placed in the centre of the oven.
   Exposure times are 10, 20, and 30 minutes respectively for the non-encapsulated phages, encapsulated phages without UV screening agent, encapsulated phages with UV screening agent, and the control NaCl buffer.
   Two hundred microlitres of each sample exposed to UV radiation were taken for enumeration.
   Prior to the enumeration performed as indicated above, as much supernatant as possible was removed, the capsules were weighed, opened with the addition of 1 mL PBS EDTA, left under agitation 5 minutes on a rotary disc agitator at moderate speed, then subjected to serial dilution to carry out enumeration according to the protocol given above.

Table 1 gives the titration results of the BPH1 phages after UV exposure times of 0, 5, 10, 20 and 30 minutes respectively. LDD is the detection limit.

TABLE 1

| | Expo | |
|---|---|---|
| | time | |
| Sample | (min) | CFU/ml |
| negative control | 0 | LDD |
| free phage | 0 | 4.33E+07 |
| wet capsules without biochar E1 | 0 | 2.06E+06 |
| wet capsules without biochar E2 | 0 | 2.89E+06 |
| wet capsules with biochar E1 | 0 | 2.89E+06 |
| wet capsules with biochar E2 | 0 | 3.67E+06 |
| negative control | 5 | LDD |
| free phage | 5 | LDD |
| wet capsules without biochar E1 | 5 | LDD |
| wet capsules without biochar E2 | 5 | LDD |
| wet capsules with biochar E1 | 5 | 4.08E+04 |
| wet capsules with biochar E2 | 5 | 3.11E+05 |
| negative control | 10 | LDD |
| free phage | 10 | LDD |
| wet capsules without biochar E1 | 10 | LDD |
| wet capsules without biochar E2 | 10 | LDD |
| wet capsules with biochar E1 | 10 | 2.06E+05 |
| wet capsules with biochar E2 | 10 | 3.50E+05 |
| negative control | 20 | LDD |
| free phage | 20 | LDD |
| wet capsules without biochar E1 | 20 | LDD |
| wet capsules without biochar E2 | 20 | LDD |
| wet capsules with biochar E1 | 20 | 1.28E+05 |
| wet capsules with biochar E2 | 20 | 4.28E+04 |
| negative control | 30 | LDD |
| free phage | 30 | LDD |
| wet capsules without biochar E1 | 30 | LDD |
| wet capsules without biochar E2 | 30 | LDD |
| wet capsules with biochar E1 | 30 | 3.61E+04 |
| wet capsules with biochar E2 | 30 | 4.61E+04 |

These results show that the titer of the phages is drastically reduced (>4 log) after 5-minute exposure to UV rays, in the same proportion as the “free” phages used as control.

A contrario, the decrease in titer is smaller for the phages encapsulated with biochar: from 1 log after 5 minutes to 2 log after 30 minute-exposure. These results clearly show that a capsule containing biochar efficiently protects the phages against inactivation.

Encapsulated and dried phages, exposed to UV rays after rehydration, gave similar results after an exposure time of 10 minutes as illustrated in Table 2.

TABLE 2

| | Expo | |
|---|---|---|
| | time | |
| Sample | (min) | CFU/ml |
| free phage | 0 | 6.00E+07 |
| dry capsules without biochar E2 | 0 | 2.61E+06 |
| dry capsules without biochar E1 | 0 | 3.11E+06 |
| dry capsules with biochar E2 | 0 | 1.78E+06 |
| dry capsules with biochar E1 | 0 | 2.00E+06 |
| free phage | 10 | 1.67E+02 |
| dry capsules without biochar E2 | 10 | 1.67E+02 |
| dry capsules without biochar E1 | 10 | 1.67E+02 |
| dry capsules with biochar E2 | 10 | 1.22E+06 |
| dry capsules with biochar E1 | 10 | 7.33E+05 |

Tables 3 and 4 give the titration results of the 31 Dx phages after exposure times to UV rays of 0, 5, 10, 20 and 30 minutes respectively. LDD indicates the detection limit for the wet capsules and dry capsules respectively.

TABLE 3

| | | Concentration |
|---|---|---|
| | Exposure | (PFU/MI |
| sample | time | or PFU/g) |
| negative control | 0 | 0.00E+00 |
| free phage | 0 | 2.24E+09 |
| wet capsules E1 | 0 | 1.09E+08 |
| wet capsules E2 | 0 | 4.24E+07 |
| negative control | 10 | 0.00E+00 |
| free phage | 10 | 1.38E+05 |
| wet capsules E1 | 10 | 1.06E+07 |
| wet capsules E2 | 10 | 4.91E+06 |
| negative control | 20 | 0.00E+00 |
| free phage | 20 | LDD |
| wet capsules E1 | 20 | 3.60E+06 |
| wet capsules E2 | 20 | 2.02E+06 |
| negative control | 30 | 0.00E+00 |
| free phage | 30 | LDD |
| wet capsules E1 | 30 | 3.83E+06 |
| wet capsules E2 | 30 | 7.69E+05 |

TABLE 4

| | | Concentration |
|---|---|---|
| | Exposure | (PFU/MI |
| sample | time | or PFU/g) |
| negative control | 0 | 1.00E+01 |
| free phage | 0 | 5.07E+08 |
| dry capsules E1 | 0 | 3.97E+07 |
| dry capsules E2 | 0 | 3.35E+07 |
| negative control | 10 | 0.00E+00 |
| free phage | 10 | LDD |
| dry capsules E1 | 10 | 2.63E+07 |
| dry capsules E2 | 10 | 3.59E+07 |
| negative control | 20 | 0.00E+00 |
| free phage | 20 | LDD |
| dry capsules E1 | 20 | 1.76E+07 |
| dry capsules E2 | 20 | 1.60E+07 |
| negative control | 30 | 0.00E+00 |
| free phage | 30 | LDD |
| dry capsules E1 | 30 | 1.17E+07 |
| dry capsules E2 | 30 | 1.38E+07 |

Tables 5 and 6 give the titration results of the 26C phages after UV exposure times of 0, 5, 10, 20 and 30 minutes respectively. LDD indicates the detection limit for the wet capsules and dry capsules respectively.

TABLE 5

| | | Concentration |
|---|---|---|
| | Exposure | (PFU/MI |
| sample | time | or PFU/g) |
| negative control | 0 | 0.00E+00 |
| free phage | 0 | 1.56E+09 |
| wet capsules E1 | 0 | 7.94E+07 |
| wet capsules E2 | 0 | 8.21E+06 |
| negative control | 10 | 0.00E+00 |
| free phage | 10 | 2.07E+04 |
| wet capsules E1 | 10 | 3.27E+07 |
| wet capsules E2 | 10 | 2.89E+06 |
| negative control | 20 | 0.00E+00 |
| free phage | 20 | LDD |
| wet capsules E1 | 20 | 1.16E+07 |
| wet capsules E2 | 20 | 2.22E+06 |
| negative control | 30 | 0.00E+00 |
| free phage | 30 | LDD |
| wet capsules E1 | 30 | 1.78E+07 |
| wet capsules E2 | 30 | 1.72E+06 |

TABLE 6

| | | Concentration |
|---|---|---|
| | Exposure | (PFU/MI |
| sample | time | or PFU/g) |
| negative control | 0 | 0.00E+00 |
| free phage | 0 | 1.50E+09 |
| dry capsules E1 | 0 | 1.18E+08 |
| dry capsules E2 | 0 | 9.47E+08 |
| negative control | 10 | 0.00E+00 |
| free phage | 10 | 9.07E+04 |
| dry capsules E1 | 10 | 1.94E+07 |
| dry capsules E2 | 10 | 1.13E+08 |
| negative control | 20 | 0.00E+00 |
| free phage | 20 | LDD |
| dry capsules E1 | 20 | 4.45E+08 |
| dry capsules E2 | 20 | 2.50E+08 |
| negative control | 30 | 0.00E+00 |
| free phage | 30 | LDD |
| dry capsules E1 | 30 | 4.68E+07 |
| dry capsules E2 | 30 | 4.69E+07 |

Tables 7 and 8 give the titration results of the P2 phages after UV exposure times of 0, 5, 10, 20 and 30 minutes respectively. LDD is the detection limit for the wet capsules and dry capsules respectively. NA indicates non-applicable (data not available for technical reasons).

TABLE 7

| | | Concentration |
|---|---|---|
| | Exposure | (PFU/MI |
| sample | time | or PFU/g) |
| negative control | 0 | 0.00E+00 |
| free phage | 0 | 1.92E+09 |
| wet capsules E1 | 0 | 1.10E+08 |
| wet capsules E2 | 0 | 1.24E+08 |
| negative control | 10 | 0.00E+00 |
| free phage | 10 | LDD |
| wet capsules E1 | 10 | 9.73E+06 |
| wet capsules E2 | 10 | 1.04E+07 |
| negative control | 20 | 0.00E+00 |
| free phage | 20 | LDD |
| wet capsules E1 | 20 | 3.52E+06 |
| wet capsules E2 | 20 | NA |
| negative control | 30 | 0.00E+00 |
| free phage | 30 | LDD |
| wet capsules E1 | 30 | 3.82E+06 |
| wet capsulesE2 | 30 | 2.73E+06 |

TABLE 8

| | | Concentration |
|---|---|---|
| | Exposure | (PFU/MI |
| sample | time | or PFU/g) |
| negative control | 0 | 0.00E+00 |
| free phage | 0 | 1.69E+09 |
| dry capsules E1 | 0 | 6.62E+08 |
| dry capsules E2 | 0 | 8.04E+07 |
| negative control | 10 | 0.00E+00 |
| free phage | 10 | 5.44E+04 |
| dry capsules E1 | 10 | 6.06E+08 |
| dry capsules E2 | 10 | 8.89E+07 |
| negative control | 20 | 0.00E+00 |
| free phage | 20 | LDD |
| dry capsules E1 | 20 | 8.56E+07 |
| dry capsules E2 | 20 | 7.20E+08 |
| negative control | 30 | 0.00E+00 |
| free phage | 30 | LDD |
| dry capsules E1 | 30 | 1.11E+08 |
| dry capsules E2 | 30 | 5.88E+07 |

Tables 9 and 10 give the titration results of the 31Dx phages after UV exposure times of 0, 5, 10, 20 and 30 minutes respectively. LDD is the detection limit for the wet and dry capsules respectively.

TABLE 9

| | | Concentration |
|---|---|---|
| | Exposure | (PFU/MI |
| sample | time | or PFU/g) |
| negative control | 0 | 0.00E+00 |
| free phage | 0 | 1.72E+07 |
| wet capsules E1 | 0 | 2.41E+06 |
| wet capsules E2 | 0 | 1.18E+06 |
| negative control | 10 | 0.00E+00 |
| free phage | 10 | LDD |
| wet capsules E1 | 10 | 4.09E+06 |
| wet capsules E2 | 10 | 5.06E+05 |
| negative control | 20 | 0.00E+00 |
| free phage | 20 | LDD |
| wet capsules E1 | 20 | 6.57E+04 |
| wet capsules E2 | 20 | 7.23E+04 |
| negative control | 30 | 0.00E+00 |
| free phage | 30 | LDD |
| wet capsules E1 | 30 | 1.71E+05 |
| wet capsules E2 | 30 | 2.96E+05 |

TABLE 10

| | | Concentration |
|---|---|---|
| | Exposure | (PFU/MI |
| sample | time | or PFU/g) |
| negative control | 0 | 0.00E+00 |
| free phage | 0 | 5.83E+06 |
| dry capsules E1 | 0 | 5.14E+06 |
| dry capsules E2 | 0 | 4.21E+06 |
| negative control | 10 | 0.00E+00 |
| free phage | 10 | LDD |
| dry capsules E1 | 10 | 1.75E+06 |
| dry capsules E2 | 10 | 5.65E+06 |
| negative control | 20 | 0.00E+00 |
| free phage | 20 | LDD |
| dry capsules E1 | 20 | 3.39E+06 |
| dry capsules E2 | 20 | 1.58E+06 |
| negative control | 30 | 0.00E+00 |
| free phage | 30 | LDD |
| dry capsules E1 | 30 | 1.22E+06 |
| dry capsules E2 | 30 | 6.44E+06 |

### Example 3: Protection of a Cocktail of Phages in a Capsule Comprising an UV Screening Agent

A cocktail of phages directed against the bacteria Salmonella, Salmofresh™ marketed by Intralytix Inc., was encapsulated in capsules comprising an UV screening agent. In parallel, two individual phages composing said cocktail, the phage SBA 1781 and phage SPT respectively, were encapsulated in the same type of capsules. All these phages were exposed to UV rays following the protocol given above and enumerated, with comparison between the phage cocktail or free phage and the negative control. This experiment was performed on both wet capsules and dry capsules. The results are given in Tables 11 and 12 below.

TABLE 11

| | | | | |
|---|---|---|---|---|
| expo | | | | |
| time | neg | free | wet | wet |
| (min) | contr | phage | cp E1 | cp E2 |

Salmofresh ™

| | | | | |
|---|---|---|---|---|
| 0 | LDD | 5.69E+07 | 9.20E+05 | 7.80E+05 |
| 10 | LDD | 4.20E+03 | 2.61E+05 | 3.44E+05 |
| 20 | LDD | 9.33E+01 | 1.48E+05 | 9.10E+04 |
| 30 | LDD | LDD | 9.20E+04 | 2.01E+05 |

SBA 1781

| | | | | |
|---|---|---|---|---|
| 0 | LDD | 1.96E+05 | 2.80E+02 | 2.10E+02 |
| 10 | LDD | LDD | 2.80E+02 | 1.70E+02 |
| 20 | LDD | LDD | 7.00E+01 | 4.00E+01 |
| 30 | LDD | LDD | 2.00E+01 | 1.00E+02 |

SPT1

| | | | | |
|---|---|---|---|---|
| 0 | LDD | 2.10E+07 | 4.40E+05 | 3.60E+05 |
| 10 | LDD | LDD | 1.68E+05 | 1.78E+05 |
| 20 | LDD | LDD | 2.41E+05 | 2.18E+05 |
| 30 | LDD | LDD | 1.27E+05 | 9.50E+04 |

TABLE 12

| | | | | |
|---|---|---|---|---|
| expo | | | | |
| time | neg | free | dry | dry |
| (min) | contr | phage | cp E1 | cp E2 |

Salmofresh ™

| | | | | |
|---|---|---|---|---|
| 0 | LDD | 4.90E+07 | 3.50E+05 | 4.00E+05 |
| 10 | LDD | 9.10E+03 | 1.25E+05 | 1.38E+05 |
| 20 | LDD | 3.27E+02 | 1.71E+05 | 1.62E+05 |
| 30 | LDD | 4.67E+01 | 6.50E+04 | 6.90E+04 |

SBA 1781

| | | | | |
|---|---|---|---|---|
| 0 | LDD | 3.31E+04 | 1.10E+02 | 1.00E+02 |
| 10 | LDD | LDD | 1.50E+02 | 7.00E+01 |
| 20 | LDD | LDD | 6.00E+01 | 7.00E+01 |
| 30 | LDD | LDD | 1.10E+02 | 1.10E+02 |

SPT1

| | | | | |
|---|---|---|---|---|
| 0 | LDD | 1.68E+07 | 3.70E+04 | 3.60E+04 |
| 10 | LDD | 1.40E+02 | 7.60E+04 | 2.30E+04 |
| 20 | LDD | LDD | 3.60E+04 | 1.80E+04 |
| 30 | LDD | LDD | 2.90E+04 | 3.90E+04 |

These results show a major decrease in the number of phages in the free phage control (i.e. non-encapsulated) on and after an UV exposure time of 10 minutes, and preserved viability of the capsules comprising a UV screening agent. Since said viability is demonstrated via the antibacterial action of the phages against their target bacterium, these results show the stability of the antibacterial composition of the invention.

1. An antibacterial composition comprising:
at least one bacteriophage chosen from the Podoviridae, Myoviridae and Siphoviridae, as an active agent in a liquid core,
a gelled shell encapsulating the liquid core comprising said bacteriophage and comprising dispersed colloidal particles of UV screening agent in which the UV screening agent is not a soluble antioxidant compound.

2. The antibacterial composition according to claim 1, wherein the UV screening agent is chosen from among titanium oxide, carbon black, biochar, charcoal, latex, silica, clays, and mixtures thereof.

3. The composition according to claim 1, wherein the bacteriophage targets at least one pathogenic plant bacterium.

4. The composition according to claim 3, wherein said at least one pathogenic plant bacterium is chosen from among Erwinia spp., Xanthomonas spp., Xylella spp., Pseudomonas spp., Ralstonia spp.

5. The composition according to claim 4, wherein said pathogenic plant bacterium is of the genus Erwinia.

6. The composition according to claim 1, wherein the bacteriophage targets at least one pathogenic bacterium likely to infect humans.

7. The composition according to claim 1, wherein the bacteriophage targets at least one pathogenic bacterium of animals.

8. The composition according to claim 1, wherein the bacteriophage targets at least one pathogenic bacterium likely to contaminate products of the agri-food industry.

9. Use of an antibacterial composition according to claim 1 for the treatment of plant crops, the seeds thereof or harvest products.

10. Use of the antibacterial composition according to claim 1 for soil treatment.

11. A method for preventing and/or treating plants against at least one pathogenic bacterium, comprising the steps of:
providing an antibacterial composition comprising at least one bacteriophage as an active agent against said at least one pathogenic bacterium, said bacteriophage being protected against UV rays in the core of a microcapsule comprising colloidal particles of UV screening agent dispersed in the shell encapsulating the core, wherein the UV screening agent is not a soluble antioxidant compound,
applying said composition to the above-ground parts of the plant and/or by ground spreading, or around the seeds.

12. Use of the antibacterial composition according to claim 1 for human nutrition and/or foodstuffs.

13. Use of an antibacterial composition according to claim 1 for animal nutrition and/or feed.

14. A pharmaceutical composition comprising an antibacterial composition according to claim 1.

15. A method for treating a mammal comprising administering a composition according to claim 14.

16. The composition according to claim 6, wherein the at least one pathogenic bacterium likely to infect humans is chosen from among Escherichia coli, Listeria monocytogenes, Campylobacter jejuni, Staphylococcus aureus, Clostridium perfringens or a bacterium of the genus Salmonella.

17. The composition according to claim 7, wherein the at least one pathogenic bacterium of animals is chosen from among Escherichia coli, Salmonella, Campylobacter, Staphylococcus, Flavobacterium, Yersinia, Clostridium.

18. The composition according to claim 8, wherein the at least one pathogenic bacterium likely to contaminate products of the agri-food industry is chosen from among Listeria, Salmonella, Shigella, Escherichia coli, Campylobacter jejuni.

19. Use of an antibacterial composition according to claim 7 for animal nutrition and/or feed.

20. Use of the antibacterial composition according to claim 6 for human nutrition and/or foodstuffs.
